(19) United States
(12) Patent Application Publication
(10) Pub. No.: US 2020/0085605 A1
(43) Pub. Date: Mar. 19, 2020

(54) Brace for aiding the handling of a limb of a patient

(71) Applicant: BIMA SRL, Genova (IT)

(72) Inventors: Giovanni Biggi, Genova (IT); Andrea Paolo Mancuso, Moconesi (GE) (IT)

(21) Appl. No.: 16/494,294

(22) PCT Filed: Mar. 19, 2018
(86) PCT No.: PCT/IB2018/051829

§ 371 Date: Sep. 15, 2019

(30) Foreign Application Priority Data
Mar. 20, 2017 (IT) 102017000030447

(51) Int. Cl. A61F 5/01

(52) U.S. Cl. CPC A61F 5/0125; A61F 2005/0169; A61F 2005/0179

(57) ABSTRACT

A brace for assisting the movement of a limb of a patient includes a support member, which is removably fixed to the limb while encircling the joint requiring movement assistance. The support member enables the limb to move on a plane, such that the movement of the limb identifies an internal angle and an external angle. A shock-absorbing device is also provided, which is fixed to the support member.

The present invention relates to a brace for assisting movement of a limb of a patient, which comprises a support member removably fixed to the limb, in such a position as to encircle the joint that requires movement assistance.

The support member allows the limb to move on a plane, such that the movement of the limb identifies an internal angle and an external angle.

The invention generally relates to any mobility assist device, both for patients under rehabilitations and for patients that want to improve their motor skills.

Particularly referring to users that want to improve their motor skills, the following description will show that the brace of the present invention is suitable for various uses, such as sports use, or for any use involving muscle enhancement.

Furthermore, as clearly shown by the following description, the invention relates to a brace that can be placed at any joint, i.e. heel, shoulder, knee, elbow, etc.

Preferably, but without limitation, the brace of the present invention relates to post-surgery physiotherapy devices, for recovery and/or improvement of user limb function.

Generally, these devices are braces that only allow flexion/extension of a limb, and prevent torsional movements thereof.

For example, after knee ligament tear surgery, the brace is required to hold the two bones of the joint, i.e. femur and tibia, in line, to avoid cartilage consumption, prevent torsional and latero-lateral movements and antero-posterior motion, while allowing flexion and extension.

Both for post-surgery physical therapy and geriatric treatment, the main problem is recovery of muscular force, because patients do not stress the joint with their own weight or with strenuous movements to avoid pain or complications, and the muscles proximate to the joint are thus weakened.

As used herein, “flexion” is intended to designate both open and closed kinematic chain flexion of the joint, particularly the knee, more appropriately defined as bending.

Systems are known in the art which have the purpose of limiting the weight applied to the joint, while ensuring proper movement and adequate muscle development.

These systems have the purpose of “robotizing” the limb, i.e. using electrically controlled devices that facilitate movements, while limiting the weight applied to the limb.

The use of electrical devices causes inevitable problems associated with the weight of the device, battery life and power consumption.

Furthermore, due to the presence of electric assistance devices, these prior art braces have considerable manufacturing and purchase costs, and are prone to failure, due to the complexity of their parts.

Therefore, a still unfulfilled need exists in the art for a brace that can assist and/or improve movement of users, while obviating prior art known drawbacks and ensuring flexible use, cost effectiveness and light weight.

The present invention fulfills the above purposes by providing a brace as described hereinabove, in which a at least one shock-absorbing device is fixed to the support member.

Therefore, in order to allow limb movement while relieving excess weight bearing, the brace of the present invention uses a mechanical system instead of the electrical devices of prior art braces.

The use of a mechanical system provides advantages in terms of costs, weight and easy construction and replacement of parts, as well as in terms of flexibility, and also involves particularly advantageous aspects from a functional point of view.

The use of a shock-absorbing device and its particular configuration provides an active range of the device and an inactive range of the device in which the joint is free to move.

A “negative” is thus obtained, i.e. a range of joint angles in which the limb moves without the assistance of the brace.

This is particularly advantageous, for example, during walking: the patient is free to walk without activating the shock-absorbing device and will only use it for wider movements.

This range of joint angles may be adjusted according to the position of the shock-absorbing device. This is a further advantageous feature associated with the use of a mechanical system: the assistance provided by the brace may be adjusted, and this allows adjustment of weight-bearing relief for the patient, by changing the position, extension and compression of the shock-absorbing device. For this purpose a preferred variant embodiment of the brace of the present invention provides the use of adjustment means, for adjusting the position of one and/or both ends of the shock-absorbing device.

Therefore, the position of the shock-absorbing device is a critical aspect, whereby variant embodiments will be described herein, specially related to different positions.

In a possible embodiment, at least one plate is provided, which is fixed to the support member and extends toward the internal angle.

This plate has a fixation seat, such that the shock-absorbing device is fixed by one end to the support member and by the other end to such fixation seat.

As an alternative to the above, the shock-absorbing member may have both ends fixed to the support member, with the ends located upstream and downstream from the joint respectively.

It will be appreciated from an exemplary embodiment that the ends are fixed in offset positions relative to the joint, to thereby obtain the aforementioned “negative” range. This configuration especially reduces the dimensions of the shock-absorbing device of the inventive brace.

This aspect is of critical importance, as the brace is fixed to the limbs and should not be too large, to allow natural movement of the limbs.

In a possible embodiment, the shock-absorbing device is composed of two parts, such parts sliding into each other while the joint is moving, and such parts delimiting a space in which a shock-absorbing member is accommodated.

Particularly, in order to adjust an amount of assistance of the brace, according to an improvement, compression adjustment means may be provided, for adjusting the compression of said shock-absorbing member.

Advantageously, the shock-absorbing member may comprise a spring.

The spring is not merely a construction variant selected among elastic members, but is able to change the assistance capacity of the brace, without changing the position of the shock-absorbing device.

Either linear or progressive springs may be used, to change the amount of brace assistance not only according to the spring types, but also according to the articulation degrees.

Spring compression changes according to the user's weight, in the same manner as in shock-absorber calibration.

The spring has such a configuration as to cause extension of the limb, and to be compressed during flexion, thereby opposing to such movement.

This configuration solves one of the main problems in elderly patients.

Elderly patients have not enough muscle strength to control wide flexion movements.

For example, when an old patient sits on a chair, the final part of the flexion movement is not controlled by muscles, but is likely to be similar to a fall, that places a large amount of stress on spinal discs, possibly to rupture.

The shock-absorbing device provides support to such final part of the movement, damps the “fall” on the chair and prevents spinal disc rupture.

The same results might be achieved if the shock-absorbing member comprises at least one pair of magnetic elements.

Furthermore, in a variant embodiment, the shock-absorbing member comprises a spring with magnetic elements attached at its ends.

If the shock-absorbing member is inside the shock-absorbing device, the compression adjustment means may consist, for instance, of shims placed within the aforementioned two parts that compose the shock-absorbing device.

Alternatively, the shock-absorbing member may be composed of two parts that slide into each other while the joint is moving, a shock-absorbing member being placed around the outer surface of these two parts.

Preferably, the shock-absorbing member consists of a spring.

Advantageously the outer surface of the two parts has threads, the adjustment means consisting of a ring nut that moves along the outer surface of the two parts.

As the ring nut is tightened/loosened, the length of the spring is decreased/increased and its compression is thus adjusted.

The provision of threads is also compatible with the use of two magnetic ring nuts which move toward and away from each other thereby adjusting the extension and compression force of the shock-absorbing member.

These ring nuts might be tightened/loosened along the surface of the shock-absorbing device, thereby acting as a spring.

It will be appreciated from the foregoing that the brace of the present invention, due to its characteristics as discussed above, affords adjustment of the negative range, the compression and extension range of the shock-absorbing member.

The use of the brace of the present invention is particularly advantageous for people with temporary impairment of motor skills, whose purpose is to recover limb function.

The following description of a few exemplary embodiments will show an additional advantage of the inventive brace, i.e. its adaptation to different operating requirements.

This is achieved by the provision of two or more shock-absorbing devices, arranged on one or both sides of the joint.

Likewise, the arrangement of the shock-absorbing devices relative to the plate may also change, depending on operating requirements and on the desired movement assistance.

In this respect, at least two shock-absorbing devices may be provided, each fixed by one end upstream and downstream from the joint respectively.

In a further embodiment, the longitudinal axes of the two shock-absorbing devices diverge toward the internal angle, whereas the other ends of each shock-absorbing device are fixed to two fixation seats of two respective plates.

Alternatively, the longitudinal axes of the two shock-absorbing devices may converge toward the internal angle, while the other ends of each shock-absorbing device are fixed to a plate located at the joint.

In view of the foregoing, and especially in view of the above discussed advantages of the inventive brace, it will be appreciated that the parts of such inventive brace, namely the shock-absorbing device and the plate, may be adapted to an existing support member.

Thus, the present invention also relates to a kit for assisting movement of a limb of a patient, which comprises an assistance device having means for fixation to a support member removably fixed to the limb, in such a position as to encircle the joint that requires movement assistance.

Particularly, the support member allows the limb to move on a plane, such that the movement of the limb identifies an internal angle and an external angle.

Furthermore, the assistance device comprises at least one shock-absorbing device.

In combination with such shock-absorbing device, in one embodiment, a plate may be provided for the shock-absorbing device to be fixed thereto.

The shock-absorbing device and/or the plate are constructed according to one or more of the above described characteristics.

These and other features and advantages of the present invention will appear more clearly from the following description of a few embodiments, illustrated in the annexed drawings, in which:

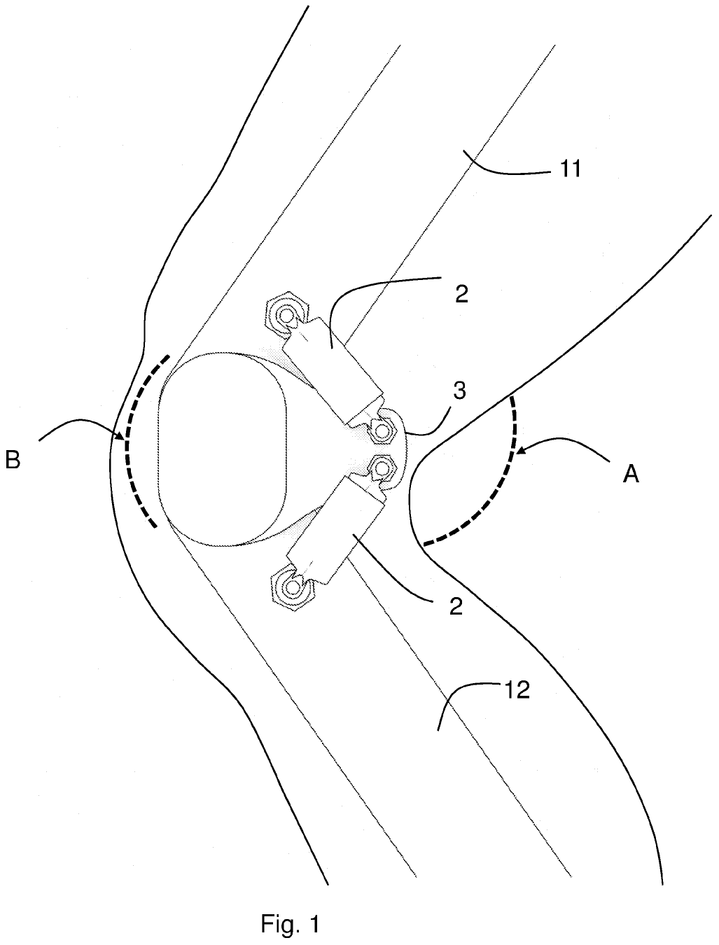

FIG. 1 shows the brace of the present invention according to a possible embodiment, as worn by a user;

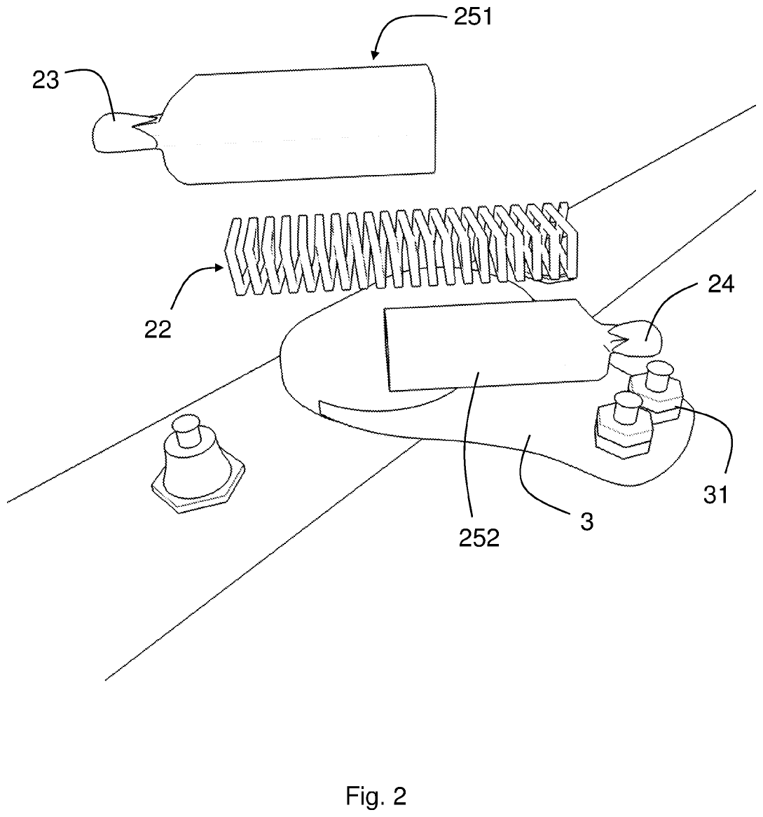

FIG. 2 shows an exploded view of the parts of the brace of the present invention according to a possible embodiment;

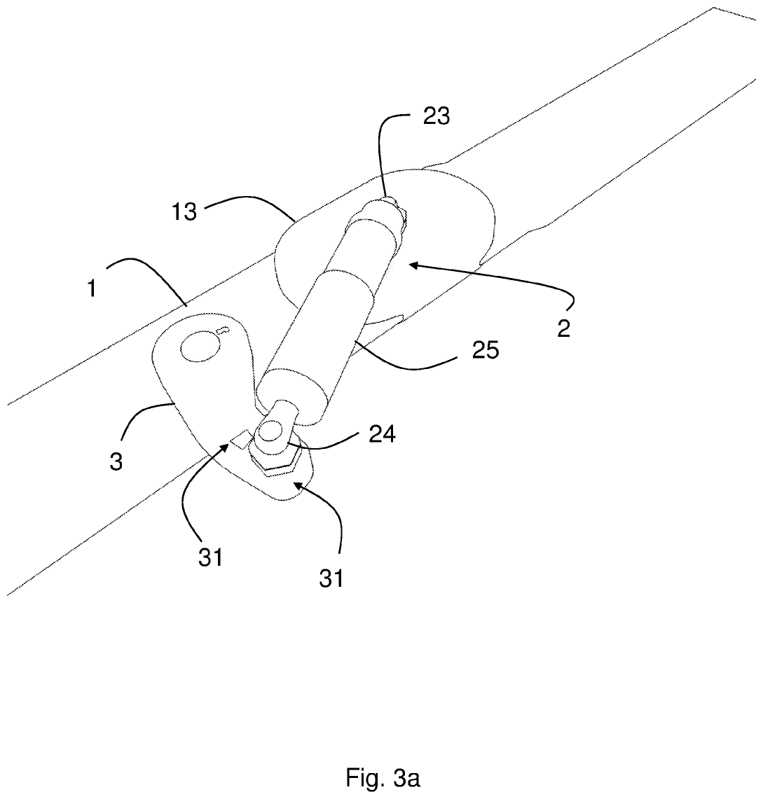

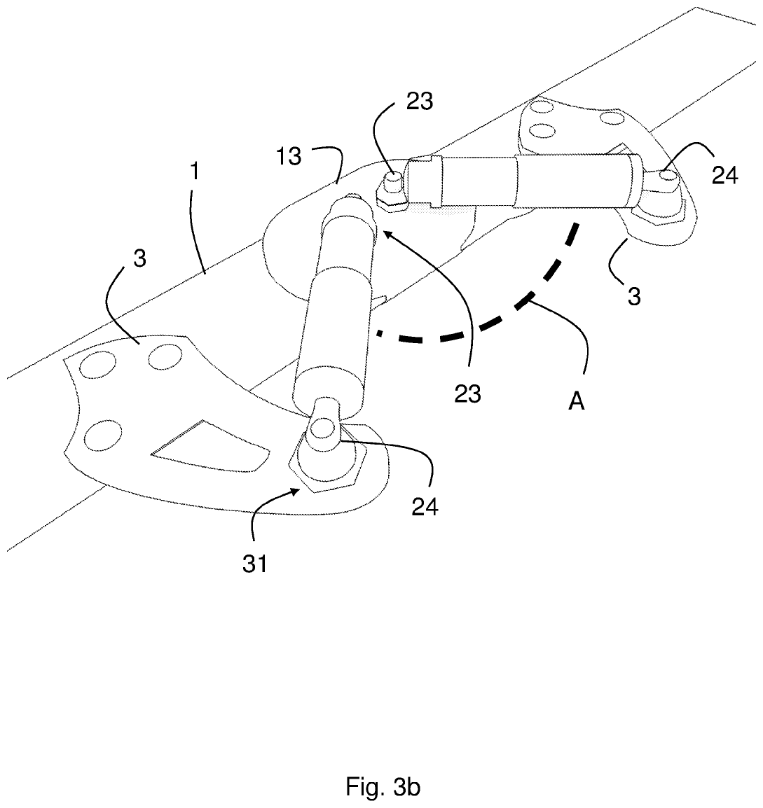

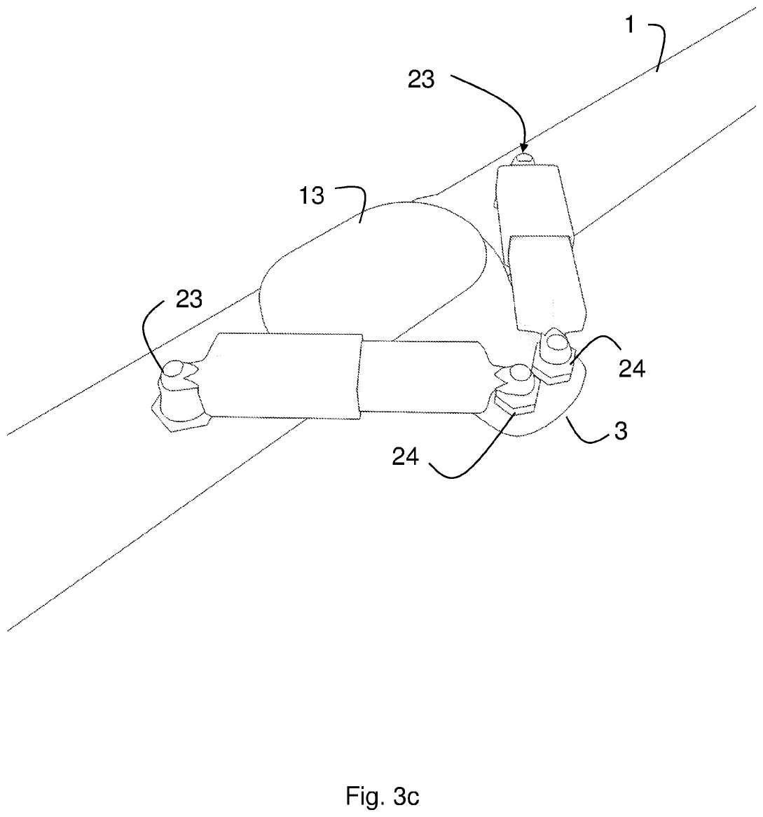

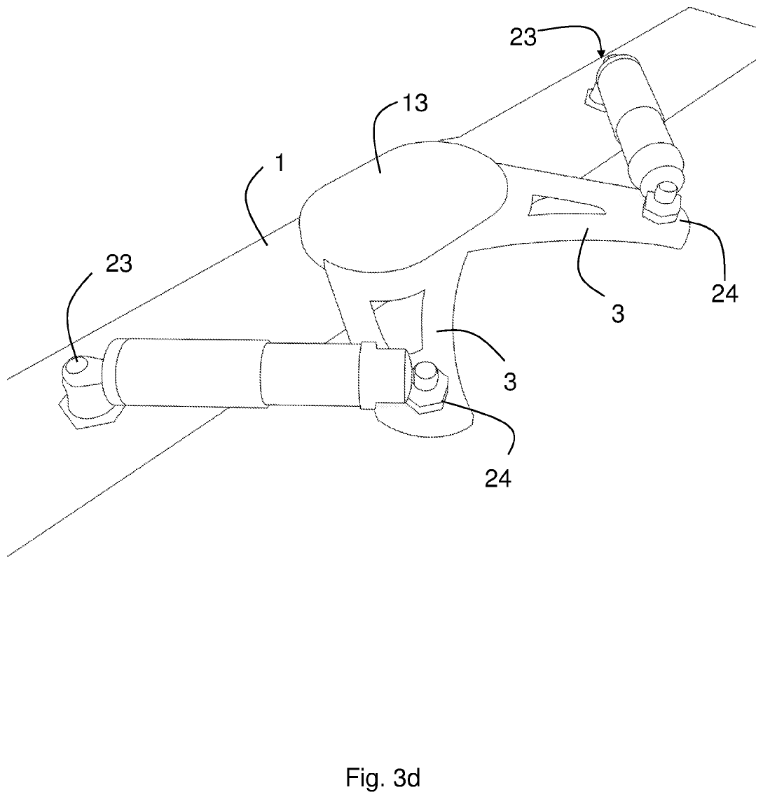

FIGS. 3a to 3d show different variant embodiments of the brace of the present invention;

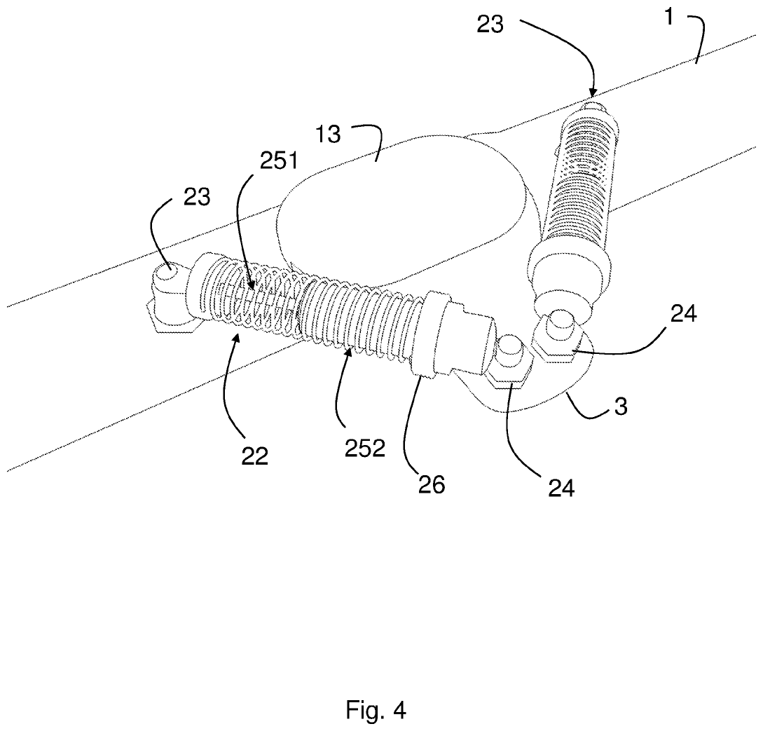

FIG. 4 shows the brace of the present invention according to a further embodiment;

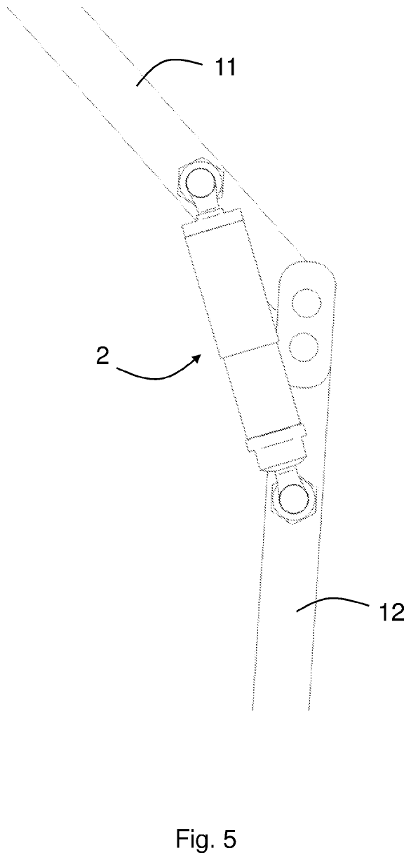

FIG. 5 shows a possible position of the shock-absorbing device that is part of the brace of the present invention;

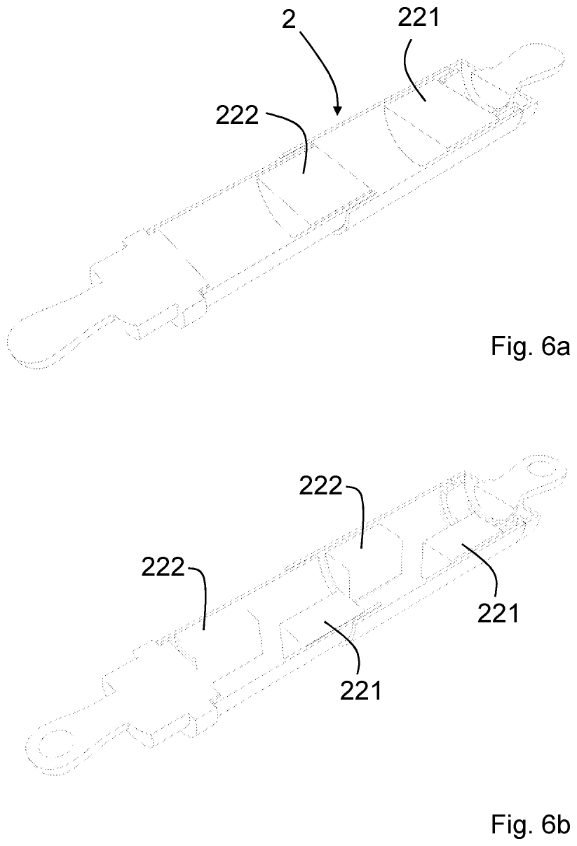

FIGS. 6a and 6b show two further embodiments of the brace of the present invention;

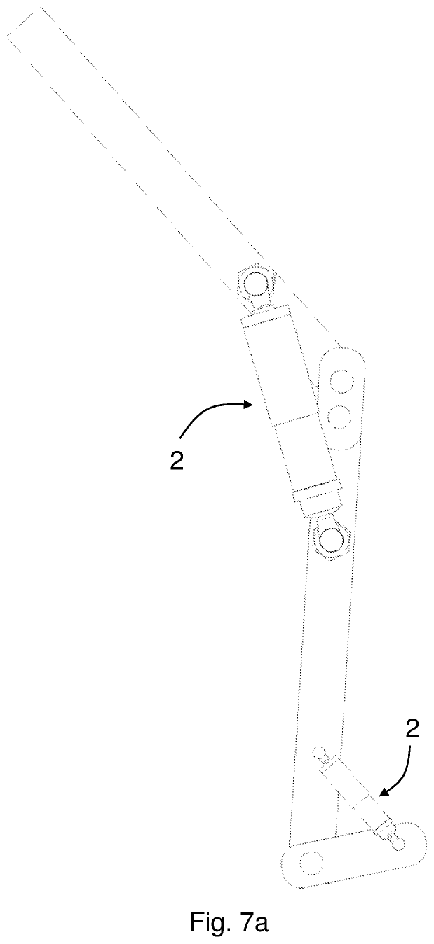

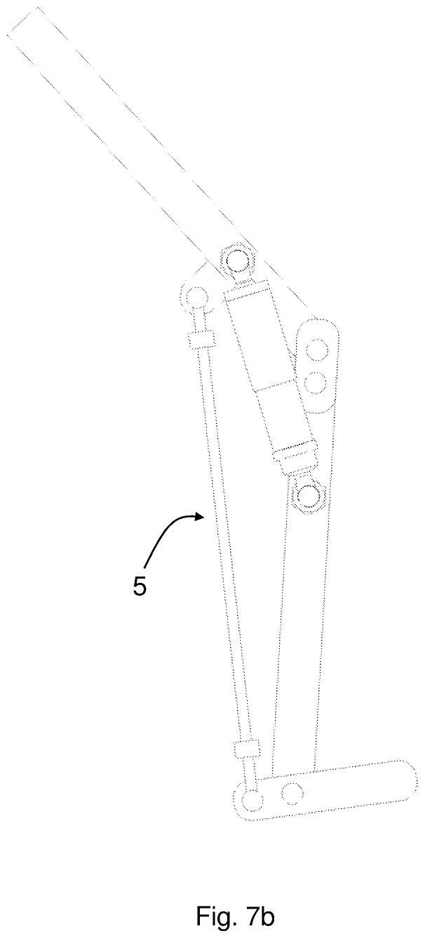

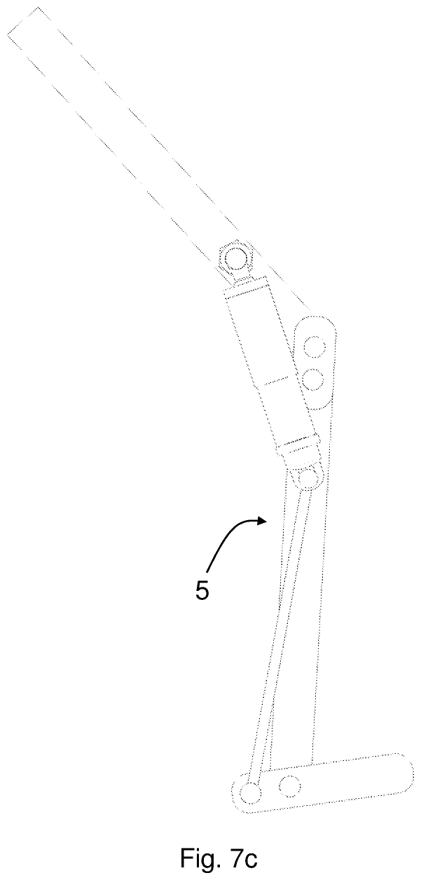

FIG. 7 shows a possible configuration of the brace of the present invention.

It shall be noted that the figures annexed to the present application depict certain embodiments of the brace of the present invention, to provide an improved understanding of its advantages and characteristics.

Therefore, such embodiments shall be intended by way of illustration and without limitation to the inventive concept of the present invention, which consists in the provision of a brace that can assist movement using parts that can be easily installed and adjusted, have a low cost and allow the brace to adapt to various operating needs.

It shall be further understood that while a knee brace is shown for simplicity herein, the invention is applicable to any joint, without requiring any change to the basic characteristics of its parts.

Particularly, FIGS. 1 and 2 show a brace of the present invention, in which the support member is a knee brace, diagrammatically shown for simplicity.

These braces are known in the art and may be four-point braces, using four anchor points, two at the tibial level and two on the thigh.

Still referring to knee braces, it will be appreciated that the various parts of the inventive brace may be used in combination with braces with splints, i.e. support members having rigid splints instead of the four anchor points, which are worn in much the same manner as socks.

Particularly referring to FIGS. 1 and 2, the inventive brace includes a support member 1, which is removably fixed to the knee, and is composed of a femoral part 11 and a tibial part 12, in such a configuration as to encircle the joint that requires movement assistance.

As shown in the figures, the support member 1 allows the limb 1 to move on a plane, such that the movement of the limb identifies an internal angle A and an external angle B.

In the case of FIG. 1, the support member allows flexion and extension of the knee, and hence movement along the sagittal plane of the user, thereby identifying an internal angle, referenced A and an external angle, referenced B.

The brace of the present invention also has at least one shock-absorbing device 2.

FIG. 2 shows an exploded view of a possible embodiment of such a shock-absorbing device 2.

At least one plate 3 is also provided, which is fixed to the support member 1 and extends toward the internal angle A.

The plate 3 also has a fixation seat 31, the shock-absorbing device 2 being fixed to such fixation seat.

The shock-absorbing device 2 is fixed by its end 23 to the support member 1 and by its end 24 to the fixation seat 31.

Both the shock-absorbing device 2 and the plate 3 extend from the support member 1 toward the internal angle A.

In view of optimizing the assistance component of the shock-absorbing device 2, the latter should lie on the same plane as the plate 3.

Further the more the plane shared by the shock-absorbing device 2 and the plate 3 is parallel to the sagittal plane, the smaller the dimensions of the brace.

According to a preferred variant embodiment, means may be provided, for adjusting the position of one and/or both of the ends 23, 24 of the shock-absorbing device 2.

These adjustment means may be formed in any manner known in the art.

For example, the ends of the shock-absorbing device 2 are fixed by means of fastening screws, such that the position of the shock-absorbing device 2 may be changed by providing adjustable seats, i.e. as slots or the like, on the plate 3 or the support member 1.

Alternatively, the ends of the shock-absorbing member may be equipped with a fastening head which engages with a corresponding fastening ball attached to the support member 1 and/or the plate 3, in much the same manner as the coupling known as “uniball”.

In the embodiment as shown in FIG. 2, the shock-absorbing device is composed of or consists of two parts 251, 252 which slide into each other while the joint is moving.

As shown in FIG. 2, the parts 251, 252 delimit a space in which a shock-absorbing member is accommodated.

The two parts 251, 252 may have any shape, i.e. a rectangular, cylindrical shape, etc.

In the particular case of FIG. 2, the shock-absorbing member is a spring 22.

The two parts 251, 252 are designed to fit one into the other and slide one upon the other, to cause extension and compression of the spring 22.

FIG. 6a shows a possible embodiment, particularly a section of the shock-absorbing device 2, in which the shock-absorbing member is composed of a pair of magnets 221, 222.

As shown in this figure, the magnets 221, 222 are located at the bottom and in the midpoint of the space identified by the two parts 251 and 252, although any position of such magnets may be provided.

Furthermore, as shown in FIG. 6b, any number of magnets may be arranged along the inner surface of the two parts, depending on operating requirements.

In the same manner as the spring 22, as the two parts 251, 252 slide as a result of the movement of the joint, the two magnets 221 and 222, exert a compression/pulling force that helps the user to move the knee.

As the magnet repel each other, they generate a thrust that opposes the movement of the parts toward each other, like the action of the spring 22.

Irrespective of whether the spring 22 or the magnets 221, 222 are used, adjustment means may be provided for adjusting the compression/pulling action of the shock-absorbing element.

In the particular case of FIGS. 2, 6a and 6b, these adjustment means may consist of shims to be placed inside the two parts 251, 252.

These shims can either adjust the stroke of the spring or decrease the distance between the two magnetic elements, to thereby increase their repulsion force.

Of course, the shock-absorbing member may also consist of a spring 22 having the two magnetic elements 221 and 222 at its ends.

The above considerations also apply to FIG. 5, in which the shock-absorbing device 2 is fixed by both ends to the support member 1, and particularly by one end to the femoral part 11 and by the other end to the tibial part 12.

As mentioned above, the shock-absorbing member 2 may be fixed to the support member in any manner known in the art.

For example, a fixation system may be provided that allows easy attachment/detachment of the shock-absorbing element 2, such as an anchor system with two firm bases, with a “fork-shaped” bracket for the ends of the shock-absorbing member to be coupled thereto.

In a possible embodiment, as shown in FIG. 3a, multiple fixation seats 31 may be provided on the plate 3, to change the position of the shock-absorbing device 2.

The same arrangement may be envisaged on the support member 1 or on a support plate 13, as shown in FIGS. 3a, 3b, for fixation of the end 23 of the shock-absorbing device 2.

An identical configuration may be provided for the end of the plate 3 that is fixed to the support member 1, such that the position of the plate 3 may be also adjusted.

FIG. 4 shows a possible embodiment of the shock-absorbing member 2, in which the shock-absorbing member 2 consists of two parts 251, 252 that slide into each other while the joint is moving.

In the case of FIG. 4, the shock-absorbing member is a cylinder with a shaft 251 sliding in a cylinder 252

A spring 22 is also provided, which is placed on the outer surface of the shock-absorbing device.

Here, the means for adjusting the compression of the spring 22 consist of a ring nut 26.

In this embodiment, the outer surface of the cylinder is formed with threads and a ring nut 26 moves along the outer surface of the cylinder.

The ring nut 26 is tightened/loosened on the threads formed on the outer surface of the cylinder and acts as a stop for the spring 22, thereby adjusting the length of the latter and the amount of movement assistance.

As described above, two ring nuts may be also provided, i.e. one ring nut fixed to the cylinder 252 and the other fixed to the shaft 251, such rings being magnetic and thus replacing the action of the spring 22, in the same manner as described concerning FIG. 6.

As mentioned above, the shock-absorbing member (whether it consists of a spring 22 or magnetic elements) is adapted to push the tibia during the leg extension movement and accompanies and supports the leg in its flexion movement, while slowing down the movement.

According to the load and type of the shock-absorbing member, the movement assistance part of the inventive brace may be changed.

Nevertheless, regardless of the type of shock-absorbing member, the particular configuration of the parts can provide a range of joint angles in which the shock-absorbing member does not assist the flexion-extension movement or assists it to a very small extent.

In the particular case of the knee, this configuration allows the user to bend the knee to walk without being assisted by the brace, the latter only being activated for joint movements having a given width.

As mentioned above, FIG. 3a only shows a possible embodiment of the brace of the present invention, i.e. the embodiment having a basic configuration of parts.

Once the support member 1 has been selected, one or more shock-absorbing devices 2 and/or one or more plates 3 may be provided, depending on operating needs.

The shock-absorbing devices may be fixed to the support member 1 either through the plate 3 or through any other prior art means.

FIGS. 3b to 3d show a few possible embodiments, and it shall be understood that these embodiments may be arranged unilaterally, i.e. on one side of the joint, or bilaterally, i.e. on both sides of the joint.

For example, particularly referring to FIG. 3a, the shock-absorbing device 2 may be provided on both sides of the joint.

If two shock-absorbing devices 2 are used on the same side, such shock-absorbing devices 2 must be each fixed by one end 23 upstream and downstream from the aforementioned joint respectively.

FIG. 3b shows a first possible configuration, with two shock-absorbing devices 2 and two plates 3, in such an arrangement that the shock-absorbing devices 2 have longitudinal axes diverging toward the internal angle A.

The other ends 24 of each shock-absorbing device 2 are fixed to two fixation seats 31 of two respective plates 3.

Alternatively, FIG. 3c shows a different configuration in which the longitudinal axes of the two shock-absorbing devices 2 converge toward the internal angle A.

Furthermore, the ends 24 of each shock-absorbing device 2 are fixed to a plate 3 located at the joint.

By holding the shock-absorbing devices with their axes converging toward the internal angle A, the distance of a shock-absorbing device 2 from the other may be changed.

The ends 24 may be arranged close to each other, like in FIG. 3c, or the ends 24 may be spaced apart, like in FIG. 3d.

Particularly, FIG. 3d shows two plates 3, although a single wider plate 3 may be provided with two fixation seats 31 for the ends 24 of the shock-absorbing devices 2.

It shall be finally noted that the shock-absorbing devices as shown in the various configurations of FIGS. 1, 5, 7 and 3a to 3d may be formed with the characteristics as described with reference to FIGS. 2, 4, 6a and 6b.

As mentioned above, the brace of the present invention is suitable for use with any joint, such as the heel.

FIG. 7a shows a particular embodiment in which two shock-absorbing members 2 are provided, which are particularly used in combination for assisting the movement of the entire lower limb, i.e. the knee and heel joints.

Here, when the foot is lifted from the ground, the shock-absorbing member 2 at heel level tends to move the foot back in its position, to cope with foot drop problems.

Alternatively, an idler arm 5 may be provided, as shown in FIGS. 7b and 7c, which allows both knee and heel joints to be exercised in combination, using a single shock-absorbing element 2, located at knee level.

Obviously, depending on where the idler arm 5 is attached, this will produce either a force that moves the foot downwards to exercise the rear muscles, or a force that pulls the foot toward the knee to exercise tibial muscles.

The invention claimed is:

1. A brace for assisting movement of a limb of a patient comprising:
a support member (1), said support member (1) being removably fixed to said limb, while encircling a joint that requires movement assistance, said support member (1) enabling said limb to move on a plane, such that a movement of said limb identifies an internal angle (A) and an external angle (B),
wherein at least one shock-absorbing device (2) is provided, which is fixed to said support member (1).

2. The brace as claimed in claim 1, further comprising means for adjusting a position of one or both ends (23, 24) of said shock-absorbing device (2).

3. The brace as claimed in claim 1, further comprising at least one plate (3), which is fixed to said support member (1), said plate (3) extending toward said internal angle (A),
said plate (3) having a fixation seat (31),
said shock-absorbing device (2) being fixed at one end (23) to said support member (1) and at another end (24) to said fixation seat (31).

4. The brace as claimed in claim 1, wherein the shock-absorbing device (2) has both ends (23, 24) fixed to said support member, with said ends located upstream and downstream from the joint respectively.

5. The brace as claimed in claim 1, wherein said shock-absorbing device (2) is composed of two parts, said parts sliding into each other while the joint is moving, said parts delimiting a space in which a shock-absorbing member is accommodated.

6. The brace as claimed in claim 5, wherein said shock-absorbing member comprises a spring (22).

7. The brace as claimed in claim 5, wherein said shock-absorbing member comprises at least one pair of magnetic elements.

8. The brace as claimed in claim 1, wherein said shock-absorbing device comprises a spring (22) with magnetic elements attached at its ends.

9. The brace as claimed in claim 1, wherein said shock-absorbing device (2) is composed of two parts, said parts sliding into each other while the joint is moving, a shock-absorbing member being provided around an outer surface of said two parts.

10. The brace as claimed in claim 9, further comprising adjustment means for adjusting a compression of said shock-absorbing member.

11. The brace as claimed in claim 10, wherein said adjustment means consist of shims adapted to be placed inside said two parts.

12. The brace as claimed in claim 10, wherein the outer surface of said two parts has threads,
said adjustment means consisting of a ring nut (26) which moves along the outer surface of said two parts.

13. The brace as claimed in claim 1, wherein at least two shock-absorbing devices (2) are provided, each (2) fixed at one end upstream and downstream from said joint respectively.

14. The brace as claimed in claim 13, wherein longitudinal axes of said two said shock-absorbing devices (2) diverge toward said internal angle (A), another end of each shock-absorbing device (2) being fixed to two fixation seats of two respective plates (3).

15. The brace as claimed in claim 14, wherein longitudinal axes of said two shock absorbing devices (2) converge toward said internal angle (A), another end of each shock-absorbing device (2) being fixed to a plate (3) located at said joint.

16. A kit for assisting a movement of a limb of a patient comprising:
an assistance device adapted to be fixed to a support member (1), said support member (1) being removably fixed to said limb, while encircling a joint that requires movement assistance,
said support member (1) enabling said limb to move on a plane, such that a movement of said limb identifies an internal angle (A) and an external angle (B),
wherein said assistance device comprises at least one shock-absorbing device (2), and
wherein said shock-absorbing device (2) is composed of two parts, said parts sliding into each other while the joint is moving, said parts delimiting a space in which a shock-absorbing member is accommodated.

17. The kit as claimed in claim 16, further comprising a plate (2) which is fixed to said support member (1), said plate (3) extending toward said internal angle (A),
said plate (3) having a fixation seat (31),
said shock-absorbing device (2) being fixed at one end (23) to said support member (1) and at another end (24) to said fixation seat (31).

18. A kit for assisting a movement of a limb of a patient comprising:
an assistance device adapted to be fixed to a support member (1), said support member (1) being removably fixed to said limb, while encircling a joint that requires movement assistance,
said support member (1) enabling said limb to move on a plane, such that a movement of said limb identifies an internal angle (A) and an external angle (B),
wherein said assistance device comprises at least one shock-absorbing device (2), and
wherein said shock-absorbing device (2) comprises a spring (22) having magnetic elements attached at its ends.
